(19) United States
(12) Patent Application Publication
(10) Pub. No.: US 2005/0010047 A1
(43) Pub. Date: Jan. 13, 2005

(54) Pteridinone derivatives as modulators of chemokine receptor activity

(76) Inventors: Roger Victor Bonnert, Loughborough (GB); Peter Alan Cage, Loughborough (GB); Simon Fraser Hunt, Loughborough (GB); Iain Alastair Stewart Walters, Loughborough (GB); Rupert Philip Austin, Loughborough (GB)

Correspondence Address: FISH & RICHARDSON P.C., 225 FRANKLIN STREET, BOSTON, MA, 02110, US

(21) Appl. No.: 10/486,503

(22) PCT Filed: Aug. 9, 2002
(86) PCT No.: PCT/GB02/03684

(30) Foreign Application Priority Data
Aug. 14, 2001 (SE) 0102716-8

(51) Int. Cl. C07D 475/04

(52) U.S. Cl. 544/258

(57) ABSTRACT

The invention provides certain pteridinone compounds of formula (I), processes and intermediates used in their preparation, pharmaceutical compositions containing them and their use in therapy.

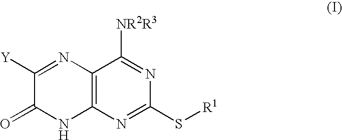

The present invention relates to certain heterocyclic compounds, processes and intermediates used in their preparation, pharmaceutical compositions containing them and their use in therapy.

Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. At the present time, the chemokine superfamily comprises three groups exhibiting characteristic structural motifs, the Cys-X-Cys (C—X—C), Cys-Cys (C—C)) and Cys-X₃-Cys (C X₃—C) families. The C—X—C and C—C families have sequence similarity and are distinguished from one another on the basis of a single amino acid insertion between the NH-proximal pair of cysteine residues. The C—X₃—C family is distinguished from the other two families on the basis of having a triple amino acid insertion between the NH-proximal pair of cysteine residues.

The C—X—C chemokines include several potent chemoattractants and activators of neutrophils such as interleukin-8 (IL8) and neutrophil-activating peptide 2 (NAP-2).

The C—C chemokines include potent chemoattractants of monocytes and lymphocytes but not neutrophils. Examples include human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

The C—X₃—C chemokine (also known as fractalkine) is a potent chemoattractant and activator of microglia in the central nervous system (CNS) as well as of monocytes, T cells, NK cells and mast cells.

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family. These receptors represent good targets for drug development since agents which modulate these receptors would be useful in the treatment of disorders and diseases such as those mentioned above.

The present invention therefore provides compounds of formula (I) and pharmaceutically acceptable salts, solvates or in vivo hydrolysable esters thereof: 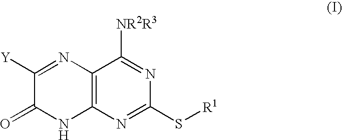
in which:
R¹ represents a C₃-C₇ carbocyclic, C₁-C₈ alkyl, C₂-C₆ alkenyl or C₂-C₆ alkynyl group, each of which may be optionally substituted by one or more substituent groups independently selected from halogen atoms, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, an aryl or heteroaryl group, which last two may themselves be optionally substituted by one or more substituents independently selected from halogen atoms, cyano, nitro, —OR⁴, —NR⁵R⁶, —CONR⁵R⁶, —COOR⁷, —NR⁸COR⁹, —SR¹⁰, —SO₂NR⁵R⁶, —NR⁸SO₂R⁹, C₁-C₆ alkyl or trifluoromethyl groups;
R² and R³ each independently represent a hydrogen atom, or a C₃-C₇ carbocyclic, C₁-C₈ alkyl, C₂-C₆ alkenyl or C₂-C₆ alkynyl group, the latter four groups may be optionally substituted by one or more substituent groups independently selected from:
(a) halogen atoms, —OR⁴, —NR⁵R⁶—CONR⁵R⁶, —COOR⁷, —NR⁸COR⁹, —SR¹⁰, —SO₂R¹⁰, —SO₂NR⁵R⁶, —NR⁸SO₂R⁹;
(b) a 3-8 membered ring optionally containing one or more atoms selected from O, S, NR⁸ and itself optionally substituted by C₁-C₃ alkyl or halogen; or
(c) an aryl group or heteroaryl group each of which may be optionally substituted by one or more substituents independently selected from halogen atoms, cyano, nitro, —OR⁴, —NR⁵R⁶, —CONR⁵R⁶, —NR⁸COR⁹, —SO₂NR⁵R⁶, —NR⁸SO₂R⁹, C₁-C₆ alkyl and trifluoromethyl groups;
R⁴ represents hydrogen or a C₁-C₆ alkyl group which may be optionally substituted by one or more substituent groups independently selected from halogen atoms, —OR¹¹, —NR⁵R⁶, or an aryl group or heteroaryl group either of which may be optionally substituted by one or more substituents independently selected from halogen atoms, cyano, nitro, —OR¹¹, —NR⁵R⁶, —CONR⁵R⁶, —NR⁸COR⁹, —SO₂NR⁵R⁶, —NR⁸SO₂, C₁-C₆ alkyl and trifluoromethyl groups; or
R⁴ represents a halogen atom, —OR¹¹, —NR⁵R⁶, or an aryl group or heteroaryl group either of which may be optionally substituted by one or more substituents independently selected from halogen atoms, cyano, nitro, —OR¹¹, —NR⁵R⁶, —CONR⁵R⁶, —NR⁸COR⁹, —SO₂NR⁵R⁶, —NR⁸SO₂R⁹, C₁-C₆ alkyl and trifluoromethyl groups;
R⁵ and R⁶ independently represent a hydrogen atom or a C₁-C₆ alkyl or phenyl group or heteroaryl group the latter three of which may be optionally substituted by one or more substituent groups independently selected from halogen atoms, phenyl, —OR¹⁴ and —NR¹⁵R¹⁶, —CONR¹⁵R¹⁶, —NR¹⁵COR¹⁶, —SONR¹⁵R¹⁶, NR¹⁵SO₂R¹⁶
   or
R⁵ and R⁶ together with the nitrogen atom to which they are attached form a 4 to 7-membered saturated heterocyclic ring system optionally containing a further heteroatom selected from oxygen and nitrogen atoms, which ring system may be optionally substituted by one or more substituent groups independently selected from phenyl, —OR¹⁴, —COOR¹⁴, NR¹⁵R¹⁶, —CONR¹⁵R¹⁶, —NR¹⁵COR¹⁶, —SONR¹⁵R¹⁶, NR¹⁵SO₂R¹⁶ or C₁-C₆ alkyl, itself optionally substituted by one or more substituents independently selected from halogen atoms and —NR¹⁵R¹⁶ and —OR¹⁷ groups;
R¹⁰ represents a C₁-C₆-alkyl or a phenyl group, either of which may be optionally substituted by one or more substituent groups independently selected from halogen atoms, phenyl, —OR¹⁷ and —NR¹⁵R¹⁶,
Y represents NR²⁰R²¹, OR⁴, SR⁴, a heteroaryl group or NR⁵R⁶ where R⁵ and R⁶ together with the nitrogen atom to which they are attached form a 4 to 7-membered saturated heterocyclic ring system optionally containing a further heteroatom selected from oxygen and nitrogen atoms, which ring system may be optionally substituted by one or more substituent groups independently selected from phenyl, —OR¹⁴, —COOR¹⁴, —NR¹⁵R¹⁶, —CONR¹⁵R¹⁶, —NR¹⁵COR¹⁶, —SONR¹⁵R¹⁶, NR¹⁵SO₂R¹⁶ or C₁-C₆ alkyl, itself optionally substituted by one or more substituents independently selected from halogen atoms and —NR¹⁵R¹⁶ and —OR¹⁷ groups;
each of R⁷, R⁸, R⁹, R¹¹, R¹⁴ R¹⁵, R¹⁶ and R¹⁷ independently represents a hydrogen atom or a C₁-C₆, alkyl, or a phenyl group;
and R²⁰ and R²¹ are defined as for R² and R³

The present invention further provides compounds of formula (I) and pharmaceutically acceptable salts or solvates thereof: 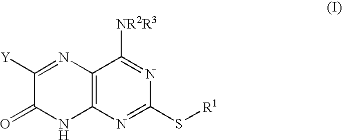
in which:
R represents a C₃-C₇carbocyclic, C₁-C₈ alkyl, C₂-C₆ alkenyl or C₂-C₆ alkynyl group, each of which may be optionally substituted by one or more substituent groups independently selected from halogen atoms, —OR⁴, —NR⁵R⁶, —CONR⁵R⁶, —COOR⁷, —NR⁸ COR⁹, —SR¹⁰, —SO₂R¹⁰, —SO₂NR⁵R⁶, —NR⁸SO₂R⁹, an aryl or heteroaryl group, which last two may themselves be optionally substituted by one or more substituents independently selected from halogen atoms, cyano, nitro, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, C₁-C₆ alkyl or trifluoromethyl groups;
R² and R³ each independently represent a hydrogen atom, or a C₃-C₇ carbocyclic, C₁-C₈ alkyl, C₂-C₆ alkenyl or C₂-C₆ alkynyl group, the latter four groups may be optionally substituted by one or more substituent groups independently selected from:
(a) halogen atoms, —OR⁴, —NR⁵R⁶—CONR⁵R⁶, —COOR⁷, —NR⁸COR⁹, —SR¹⁰, —SO₂R¹⁰, —SO₂NR⁵R⁶, —NR⁸SO₂R⁹;
(b) a 3-8 membered ring optionally containing one or more atoms selected from O, S, NR⁸ and itself optionally substituted by C₁-C₃-alkyl or halogen; or
(c) an aryl group or heteroaryl group each of which may be optionally substituted by one or more substituents independently selected from halogen atoms, cyano, nitro, —OR⁴, —NR⁵R⁶, —CONR⁵R⁶, —NR⁸COR⁹, —SO₂NR⁵R⁶, —NR⁸SO₂R⁹, C₁-C₆ alkyl and trifluoromethyl groups;
R⁴ represents hydrogen or a C₁-C₆ alkyl group the latter of which may be optionally substituted by one or more substituent groups independently selected from halogen atoms, —OR¹¹, —NR⁵R⁶, or an aryl group or heteroaryl group either of which may be optionally substituted by one or more substituents independently selected from halogen atoms, cyano, nitro, —OR¹¹, —NR⁵R⁶, —CONR⁵R⁶, —NR⁸COR⁹, —SO₂NR⁵R⁶, —NR⁸SO₂R⁹, C₁-C₆ alkyl and trifluoromethyl groups;
R⁵ and R⁶ independently represent a hydrogen atom or a C₁-C₆ alkyl or phenyl group the latter two of which may be optionally substituted by one or more substituent groups independently selected from halogen atoms, phenyl, —OR¹⁴ and —NR¹⁵R¹⁶, —CONR¹⁵R¹⁶, —CONR¹⁵R¹⁶, —NR¹⁵COR¹⁶, SONR¹⁵R¹⁶, NR¹⁵SO₂R¹⁶
   or
R⁵ and R⁶ together with the nitrogen atom to which they are attached form a 4- to 7-membered saturated heterocyclic ring system optionally containing a further heteroatom selected from oxygen and nitrogen atoms, which ring system may be optionally substituted by one or more substituent groups independently selected from phenyl, —OR¹⁴, —COOR¹⁴, —NR¹⁵R¹⁶, —CONR¹⁵R¹⁶, NR¹⁵COR¹⁶, SONR¹⁵R¹⁶, NR¹⁵SO₂R¹⁶ or C₁-C₆ alkyl, itself optionally substituted by one or more substituents independently selected from halogen atoms and —NR¹⁵R¹⁶ and —OR¹⁷ groups;
R¹⁰ represents a C₁-C₆-alkyl or a phenyl group, either of which may be optionally substituted by one or more substituent groups independently selected from halogen atoms, phenyl, —OR¹⁷ and —NR¹⁵R¹⁶,
Y is NR²⁰R²¹, OR⁴ or SR⁴;
each of R⁷, R⁸, R⁹, R¹¹, R¹², R¹³, R¹⁴ R¹⁵, R¹⁶ , R¹⁷, R¹⁸ and R¹⁹ independently represents a hydrogen atom or a C₁-C₆, alkyl, or a phenyl group;
and R²⁰ and R²¹ are defined as for R² and R³.

In the context of the present specification, unless otherwise indicated, the term alkyl includes both straight-chain and branched-chain alkyl groups. However references to individual alkyl groups such as “propyl” are specific for the straight chain version only and references to individual branched-chain alkyl groups such as t-butyl are specific for the branched chain version only. Examples of C₁-C₃ alkyl include methyl, ethyl, propyl. Examples of C₁-C₆ alkyl include the examples of C₁-C₃ alkyl and additionally butyl, t-butyl, pentyl, 2methylbutyl and hexyl. Examples of C₁-C₈ alkyl include the examples of C₁-C₆alkyl and additionally heptyl, 2-ethyl-3-methylbutyl and octyl. An analogous convention applies to other terms such as alkenyl and alkynyl. For example C₂-C₆ alkenyl includes vinyl, allyl, 1-propenyl, 2-butenyl, 2methylbut-2-enyl, and 4-hexenyl. Examples of C₂-C₆ alkynyl include ethynyl, 1-propynyl, 2-propynyl and 1-methylpent-2ynyl.

C₃-C₇ carbocyclic is a saturated, partially saturated or unsaturated ring system containing 3 to 7 ring carbon atoms. C₃-C₇ carbocyclic groups include cyclobutyl, cyclopentyl, cyclopentenyl, cyclohexyl and cyclohexenyl.

Examples of 4 to 7-membered saturated heterocyclic ring systems optionally containing a further heteroatom selected from oxygen and nitrogen atoms include azetidinyl, pyrrolidinyl, piperidinyl, morpholinyl and piperazinyl.

Aryl groups include phenyl and naphthyl. Heteroaryl is defined as a 5- or 6membered aromatic ring containing one or more heteroatoms selected from N, S, O. Examples include pyridine, pyrimidine, thiazole, oxazole, pyrazole, imidazole, furan.

Further examples include pyridine, pyrimidine, thiazole, oxazole, pyrazole, imidazole, furan, triazole and thiadiazole.

Halogen atoms include fluorine, chlorine, bromine and iodine. Preferred halogen atoms are fluorine and chlorine.

Where a group is substituted or optionally substituted by one or more substitutents it is to be understood that this definition includes all substituents being chosen from one of the specified groups or the substituents being chosen from two or more of the specified groups. Preferably one or more means 1, 2 or 3. One or more may also mean 1 or 2. Where a ring contains or optionally contains one or more atoms, preferably it contains 1, 2, 3 or 4 atoms.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

The invention further encompasses all solvated forms of compounds of formula (I) and salts thereof.

Preferred values of R¹, R², R³ and Y are as follows. Such values may be used where appropriate with any of the definitions, claims or embodiments defined hereinbefore or hereinafter.

Suitably the group R¹ represents a C₃-C₇ carbocyclic, C₁-C₈ alkyl, C₂-C₆ alkenyl or C₂-C₆ alkynyl group, each of which may be optionally substituted by one or more substituent groups independently selected from halogen atoms, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, an aryl or heteroaryl group both of which can be optionally substituted by one or more substituents independently selected from halogen atoms, cyano, nitro, —OR⁴, NR⁵R⁶, —CONR⁵R⁶, —COOR⁷, —NR⁸COR¹⁰, —SR¹⁰, —SO₂R¹⁰, —SO₂NR⁵R⁶, —NR⁸SO₂R¹⁰, C₁-C₆ alkyl or trifluoromethyl groups. Particularly advantageous compounds of formula (I) are those in which R¹represents an optionally substituted benzyl group. More preferably R¹ represents benzyl or benzyl substituted by one or more C₁-C₆ alkyl, C₁-C₆ alkoxy, or halogen atoms, in particular benzyl substituted by two halogen atoms.

Preferably one of R² and R³ is hydrogen and the other is C₁-C₈ alkyl substituted by hydroxy and one or more methyl or ethyl groups. More preferably one of R² and R³ is hydrogen and the other is CH(CH₃)CH₂OH, CH(Et)CH₂OH, C(CH₃)₂CH₂OH or CH(CH₂OH)₂. When one of R² and R³ is hydrogen and the other is CH(CH₃)CH₂OH or CH(Et)CH₂OH the resulting compounds of formula (I) are preferably in the form of the (R) isomer. Most preferably one of R² and R³ is hydrogen and the other is CH(CH₃)CH₂OH.

Preferably Y represents —NR²⁰R²¹, —OR⁴, SR⁴, a heteroaryl group or —NR⁵R⁶ where R⁵ and R⁶ together with the nitrogen atom to which they are attached form a 4 to 7-membered saturated heterocyclic ring system optionally containing a further heteroatom selected from oxygen and nitrogen atoms, which ring system may be optionally substituted by one or more substituent groups independently selected from —OH, —NH₂ or C₁-C₄ allyl.

Preferably one of R²⁰ and R²¹ is hydrogen or methyl and the other is a C₃-C₇carbocyclic substituted by hydroxy or it is C₁-C₄alkyl substituted by —OR⁴, heteroaryl optionally substituted by methyl, or a 3-8 membered ring optionally containing one or more atoms selected from O, S and NR⁸.

Preferably R⁴ represents hydrogen or a C₁-C₆ alkyl group the latter of which may be optionally substituted by —NR⁵R⁶ or an heteroaryl group which may be optionally substituted by one or more substituents independently selected from halogen atoms, cyano, nitro, —OR¹¹, —NR⁵R⁶₃, —CONR⁵R⁶, —NR⁸COR⁹, —SO₂NR⁵R⁶, —NR⁸SO₂R⁹, C₁-C₆ alkyl and trifluoromethyl groups; or
R⁴ represents a heteroaryl group which may be optionally substituted by one or more substituents independently selected from —OH and methyl.

Preferably one of R⁵ and R⁶ is hydrogen and the other is C₁-C₆alkyl or a heteroaryl group; or together with the nitrogen atom to which they are attached R⁵ and R⁶ form a 4- to 7-membered saturated heterocyclic ring system optionally containing a further heteroatom selected from oxygen and nitrogen atoms, which ring system may be optionally substituted by one or more substituent groups independently selected from —OH, —NH₂ or C₁-C₄ alkyl.

A preferred class of compound is of formula (I) in which;
R¹ represents benzyl or benzyl substituted by one or more C₁-C₆ alkyl, C₁-C₆ alkoxy, or halogen atoms;
R² represents hydrogen;
R³ represents C₁-C₈ alkyl substituted by hydroxy and one or more methyl or ethyl groups;
Y represents —NR²⁰R²¹, —OR⁴, —SR⁴, a heteroaryl group or —NR⁵R⁶;
R²⁰ represents hydrogen or methyl;
R²¹ represents a C₃-C₇carbocyclic substituted by hydroxy; or C₁-C₄alkyl substituted by —OR⁴, heteroaryl (optionally substituted by methyl), or a 3-8 membered ring optionally containing one or more atoms selected from O, S and NR⁸;
R⁴ represents hydrogen or a C₁-C₆ alkyl group optionally substituted by —NR⁵R⁶ or an heteroaryl group optionally substituted by one or more substituents independently selected from halogen atoms, cyano, nitro, —OR¹¹, —NR⁵R⁶, —CONR⁵R⁶, —NR⁸COR⁹, —SO₂NR⁵R⁶, —NR⁸SO₂R⁹, C₁-C₆ alkyl and trifluoromethyl groups; or R⁴represents a heteroaryl group optionally substituted by one or more substituents independently selected from —OH and methyl;
R⁵ represents hydrogen;
R⁶ represents C₁-C₆alkyl or a heteroaryl group;
or R⁵ and R⁶ together with the nitrogen atom to which they are attached form a 4 to 7-membered saturated heterocyclic ring system optionally containing a further heteroatom selected from oxygen and nitrogen atoms, which ring system may be optionally substituted by one or more substituent groups independently selected from —OH, —NH₂ or C₁-C₄ alkyl;
R⁸ represents a hydrogen atom or a C₁-C₆ alkyl or a phenyl group;
R⁹ represents a hydrogen atom or a C₁-C₆ alkyl or a phenyl group; and
R¹¹ represents a hydrogen atom or a C₁-C₆ alkyl or a phenyl group.

Another preferred class of compound is of formula (I) in which;
R¹ represents benzyl substituted by two halogen atoms;
R² represents hydrogen;
R³ represents CH(CH₃)CH₂OH, CH(Et)CH₂OH, C(CH₃)₂CH₂OH or CH(CH₂OH)₂;
Y represents —NR²⁰R²¹, —OR⁴, —SR⁴, a heteroaryl group or —NR⁵R⁶;
R²⁰ represents hydrogen or methyl;
R²¹ represents a C₃-C₇carbocyclic substituted by hydroxy; or C₁-C₄alkyl substituted by —OR⁴, heteroaryl (optionally substituted by methyl), or a 3-8 membered ring optionally containing one or more atoms selected from O, S and NR⁸;
R⁴ represents hydrogen or a C₁-C₆ alkyl group optionally substituted by —NR⁵R⁶ or an heteroaryl group optionally substituted by one or more substituents independently selected from halogen atoms, Cyano, nitro, —OR¹¹, —NR⁵R⁶, —CONR⁵R⁶, —NR⁸COR⁹, —SO₂NR⁵R⁶, —NR⁸SO₂R⁹, C₁-C₆ alkyl and trifluoromethyl groups; or R⁴ represents a heteroaryl group optionally substituted by one or more substituents independently selected from —OH and methyl;
R⁵ represents hydrogen;
R⁶ represents C₁-C₆alkyl or a heteroaryl group;
or R⁵ and R⁶ together with the nitrogen atom to which they are attached form a 4- to 7-membered saturated heterocyclic ring system optionally containing a further heteroatom selected from oxygen and nitrogen atoms, which ring system may be optionally substituted by one or more substituent groups independently selected from —OH, —NH₂ or C₁-C₄ alkyl;
R⁸ represents a hydrogen atom or a C₁-C₆ alkyl or a phenyl group;
R⁹ represents a hydrogen atom or a C₁-C₆ alkyl or a phenyl group; and
R¹¹ represents a hydrogen atom or a C₁-C₆ alkyl or a phenyl group.

Another preferred class of compound is of formula (I) in which;
R¹ represents benzyl substituted by two fluorine atoms;
R² represents hydrogen;
R³ represents CH(CH₃)CH₂OH;
Y represents (2-hydroxyethyl)amino, (phenylmethyl)amino, amino, 1H-imidazolyl, (1-methyl-1-imidazolyl)thio, methoxy, (3-pyridylmethyl)amino, [(5-methyl-2-furanyl)methyl]amino, 3,5-dimethyl-1-piperazinyl, N-methyl-N-[(3-methyl-5-isoxazolyl)methyl]amino, [2-(2-pyrimidinylamino)ethyl]amino, 4morpholinyl, [2-(4-morpholinyl)ethyl]amino, (2-methoxyethyl)amino, (2-furanylmethyl)amino, 1-azetidinyl, [(5-methylpyrazinyl)methyl]amino, [2-(2-furanyl)ethyl]amino, [3-(4morpholinyl)propyl]amino, [3-methyl-5-isoxazolyl)methyl]amino, 3-hydroxy-1-pyrrolidinyl, (2-furanylmethyl)thio, (2-hydroxypropyl)amino, [2-(dimethylamino)ethyl]thio, (2-hydroxypropyl)amino, (3-hydroxypropyl)amino, N-(2-hydroxyethyl)-N-methylamino, (5-hydroxy-4-methyl4H-1,2,4-triazol-3-yl)thio, (4-hydroxycyclohexyl)amino, 1,3,4-thiazol-2-ylthio, [4-hydroxy-2-cyclopenten-1-yl]amino, 3-hydroxy-1-pyrrolidinyl, 3-hydroxy-3-methyl-1-azetidinyl, 3-amino-1-pyrrolidinyl and (2-aminoethyl)thio.

Particularly preferred compounds of the invention include:
2-[[(2,3-difluorophenyl)methyl]thio]-6-[(2-hydroxyethyl)amino]-4-[[(1R)-2-hydroxy-1-methylethyl]amino]-7(8H)pteridinone;
2[[(2,3-difluorophenyl)methyl]thio]-4-[[(1R)-2-hydroxy-1-methylethyl]amino]-6-[(phenylmethyl)amino]-7(8H)pteridinone;
2-[[(2,3-Difluorophenyl)methyl]thio-4-[[(1R)-2-hydroxy-1-methylethyl]amino]-6,7-pteridinedione;
6-amino-2-[[(2,3-difluorophenyl)methyl]thio]-4-[[(1R)-2-hydroxy-1-methylethyl]amino]-7(8H)-pteridinone;
2-[[2,3-difluorophenyl)methyl)thio]-4-[[(1R)-2-hydroxy-1-methylethyl]amino]-6-(1H-imidazol-1-yl)-7(8H)pteridinone;
2-[[2,3-difluorophenyl)methyl)thio]-4-[[(1R)-2-hydroxy-1-methylethyl]amino]-[(1-methyl-1-imidazol-2-yl)thio]-7(8H)-pteridinone;
2-[[2,3-difluorophenyl)methyl)thio]-4-[[(1R)-2-hydroxy-1-methylethyl]amino]6-methoxy-7(8H)-pteridinone;
2-[[(2,3-difluorophenyl)methyl]thio]-4-[[(1R)-2-hydroxy-1-methylethyl]amino]-6-[(3-pyridinylmethyl)amino]-7(8H)-pteridinone;
2-[[(2,3-difluorophenyl)methyl]thio]-4-[[(1R)-2-hydroxy-1-methylethyl]amino]-6-[[(5-methyl-2-furanyl)methyl]amino]-7(8H)-pteridinone;
2-[[(2,3-difluorophenyl)methyl]thio]-6-[(3R,5S)-3,5-dimethyl-1-piperazinyl]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino)-6-methyl[(3-methyl-5-isoxazolyl)methyl]amino]-7(8H)-pteridinone;
2-[[(2,3-difluorophenyl)methyl]thio]-4-[[(1R)-2-hydroxy-1-methylethyl]amino]-6-[[2-(2-pyrimidinylamino)ethyl]amino]-7(8H)-pteridinone;
2-[[(2,3 difluorophenyl)methyl]thio]-4-[[(1R)-2-hydroxy-1-methylethyl]amino]-6-(4-morpholinyl)7(8H)-pteridinone;
2[[(2,3-difluorophenyl)methyl]thio]-4-[[(1R)-2-hydroxy-1-methylethyl]amino]-6-[[2-(4-morpholinyl)ethyl]amino]-7(8H)-pteridinone;
2-[[(2,3-difluorophenyl)methyl]thio]-4-[[(1R)-2-hydroxy-1-methylethyl]amino]-6-[(2-methoxyethyl)amino]-7(8H)pteridinone;
2-[[(2,3-difluorophenyl)methyl]thio]-6-[(2-furanylmethyl)amino]-4-[[(1R)-2-hydroxy-1-methylethyl]amino]-7(8H)-pteridinone;
6(1-azetidinyl)-2-[[(2,3-difluorophenyl)methyl]thio]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[[(5-methylpyrazinyl)methyl]amino]-7(8H)-pteridinone;
2-[[(2,3-difluorophenyl)methyl]thio]-6-[[2-(2-furanyl)ethyl]amino]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[[3-(4-morpholinyl)propyl]amino]-7(8H)-pteridinone;
2-[[(2,3-difluorophenyl)methyl]thio]-4-[[(1R)-2hydroxy-1-methylethyl]amino)-6-[[(3-methyl-5-isoxazolyl)methyl]amino]-7(8H)-pteridinone;
2-[[(2,3-difluorophenyl)methyl]thio]-4-[[(1R)-2-hydroxy-1-methylethyl]amino]-6-[(3S)-3-hydroxy-1pyrrolidinyl]-7(8H)-pteridinone;
2-[[(2,3-difluorophenyl)methyl]thio]-6-[(2-furanylmethyl)thio]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[(2-hydroxypropyl)amino]-7(8H)pteridinone;
2-[[(2,3-difluorophenyl)methyl]thio]-6-[[2-(dimethylamino)ethyl]thio]-4-[[(1R)-2-hydroxy-1-methylethyl]amino]-7(8H)pteridinone;
2-[[(2,3-difluorophenyl)methyl]thio]-4-[[(1R)-2-hydroxy-1-methylethyl]amino]-6-[[(2S)-2-hydroxypropyl]amino]-7(8H)-pteridinone;
2-[[(2,3-difluorophenyl)methyl]thio]-4-[[(1R)-2-hydroxy-1-methylethyl]amino]-6-[(3-hydroxypropyl)amino]-7(8H)-pteridinone;
2[[(2,3-difluorophenyl)methyl]thio]-6-[(2-hydroxyethyl)methylamino]-4-[[(1R)-2-hydroxy-1-methylethyl]amino]-7(8H)pteridinone;
2-[[(2,3-difluorophenyl)methyl]thio]-4-[[(1R)-2-hydroxy-1-methylethyl]amino]-6-[(5-hydroxy4methyl4H-1,2,4triazol-3-yl)thio]-7(8H)-pteridinone;
2-[[(2,3 difluorophenyl)methyl]thio]-6-[(4hydroxycyclohexyl)amino]-4-[[(1R)-2hydroxy-1-methylethyl]amino]-7(8H)-pteridinone;
2-[[(2,3-difluorophenyl)methyl]thio]-4-[[(1R)-2-hydroxy-1-methylethyl]amino]-6-(1,3,4-thiadiazol-2-ylthio)-7(8H)-pteridinone;
2[[(2,3-difluorophenyl)methyl]thio]-6-[[(1S,4R)-4-hydroxy-2-cyclopenten-1-yl]amino]-4-[[(1R)-2-hydroxy-1-methylethyl]amino]-7(8H)-pteridinone;
2-[[(2,3-difluorophenyl)methyl]thio]-4-[(1R)-2-hydroxy-1-methylethyl]amino]-6-[(3R)-3-hydroxy-1-pyrrolidinyl]-7(8H)-pteridinone;
2-[[(2,3-difluorophenyl)methyl]thio]-6-(3-hydroxy-3-methyl-1-azetidinyl)-4-[[(1R)-2-hydroxy-1-methylethyl]amino]-7(8H)pteridinone;
6-[(3S)-3-amino-1-pyrrolidinyl]-2-[[(2,3-difluorophenyl)methyl]thio]-4-[[(1R)-2-hydroxy-1-methylethyl]amino]-7(8H)-pteridinone; and
6-[(2-aminoethyl)thio]-2-[[(2,3difluorophenyl)methyl]thio]-4-[[(1R)-2-hydroxy-1-methylethyl]amino]-7(8H)-pteridinone.

According to the invention there is also provided a process for the preparation of:—
(a) a compound of formula (I) where Y is NR²⁰R²¹ which comprises treatment of a compound of formula (IIA) where R¹, R² and R³ are as defined in formula (I) or are protected derivatives thereof and L is a leaving group such as bromo with an amine HNR²⁰R²¹, 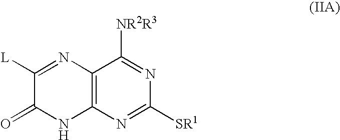
(b) a compound of formula (I) where Y is OR⁴ which comprises treatment of a compound of formula (IIA) where R¹, R² and R³ are as defined in formula (I) or are protected derivatives thereof and L is a leaving group such as bromo with an alcohol R⁴OH,
(c) a compound of formula (I) where Y is SR⁴ which comprises treatment of a compound of formula (IIA) where R¹, R² and R³ are as defined in formula (I) or are protected derivatives thereof and L is a leaving group such as bromo with a thiol R⁴SH,
(d) a compound of formula (I) where Y is NR⁵R⁶which comprises treatment of a compound of formula (IIA) where R¹, R² and R³ are as defined in formula (I) or are protected derivatives thereof and L is a leaving group such as bromo with an amine H NR⁵R⁶
(e) a compound of formula (I) where Y is a heteroaryl group which comprises treatment of a compound of formula (IIA) where R¹, R² and R³ are as defined in formula (I) or are protected derivatives thereof and L is a leaving group such as bromo with a heteroarene.
(f) a compound of formula (I) where Y is OH which comprises treatment of a compound of formula (IIB): 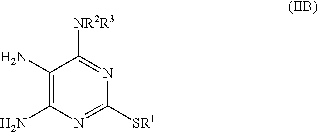
   where R¹, R² and R³ are as defined in formula (I) or are protected derivatives thereof with diethyl oxalate, or
(g) a compound of formula (I) where Y is NH₂ which comprises treatment of a compound of formula (IIB) where R¹, R² and R³ are as defined in formula (I) or are protected derivatives
thereof with iminomethoxy-acetic acid, methyl ester hydrochloride, and optionally thereafter process (a), (b), (c), (d) or (e) and in any order.
   removing any protecting groups
   forming a pharmaceutically acceptable salt, solvate or in vivo hydrolysable ester.

The reaction of compounds of formula (IIA) with an amine HNR²⁰R²¹ may be performed in a solvent such as N-methylpyrrolidinone at a temperature between 0° C. and 150° C. in the presence of a base such as N,N-diisopropylethylamine.

The reaction of compounds of formula (IIA) with an alcohol R⁴OH may be performed using the alcohol R⁴OH as solvent at a temperature between 0° C. and 150° C. in the presence of a base such as butyllithium.

The reaction of compounds of formula (IIA) with a thiol R⁴SH may be performed in a solvent such as DMSO at a temperature between 0° C. and 150° C. in the presence of a base such as potassium tert-butoxide.

The reaction of compounds of formula (IIA) with an amine HNR⁵R⁶ may be performed in a solvent such as N-methylpyrrolidinone at a temperature between 0° C. and 150° C. in the presence of a base such as N,N-diisopropylethylamine.

The reaction of compounds of formula (IIA) with a heteroarene may be performed in a solvent such as DMSO at a temperature between 0° C. and 100° C. in the presence of a base such as potassium tert-butoxide.

The reaction of compounds of formula (IIB) with diethyl oxalate may be performed in the absence of solvent at a temperature between 50° C. and 200° C.

The reaction of compounds of formula (IIB) with iminomethoxy-acetic acid, methyl ester hydrochloride may be performed in ethanol in the presence of base such as N,N-diisopropylethylamine at a temperature between 0° C. and 150° C.

Compounds of formula (IIA) where R¹, R² and R³ are as defined in formula (I) and L is bromo may be prepared from compounds of formula (IIA) where R¹, R² and R³ are as defined above and L is hydrogen by treatment with bromine in a solvent such as acetonitrile at a temperature between 0° C. and 100° C.

Compounds of formula (IIA) where R¹, R² and R³ are as defined in formula (I) and L is hydrogen may be prepared from compounds of formula (III) where R¹ is as defined above and X is a leaving group such as bromo by treatment with an amine HNR²R³. The reaction may be performed in a solvent such as N-methylpyrrolidinone at a temperature between 0° C. and 150° C. in the presence of a base such as N,N diisopropylethylamine.

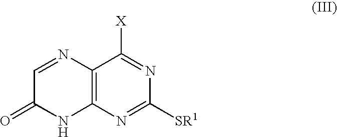

Compounds of formula (III) where R¹ is as defined in formula (I) and X is a leaving group such as bromo may be prepared by treating a compound of formula (III) where R¹ is as defined above and X is NH² with a diazotizing agent such as isoamyl nitrite in the presence of a halogenating agent such as bromoform. The reaction may be performed in a solvent such as DMSO at a temperature between 0° C. and 150° C.

Compounds of formula (III) where R¹ is as defined in formula (I) and X is NH₂ may be prepared by treatment of a compound of formula (IV): 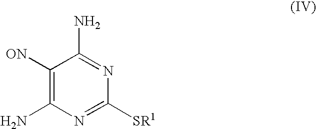
where R¹ is as defined above with triethyl phosphonoacetate in the presence of a base such as butyllithium. The reaction may be carried out in a solvent such as DMF at a temperature between 0° C. and 100° C.

Compounds of formula (IV) where R¹ is as defined in formula (I) may be prepared by treating a compound of formula (V) where R¹ is as defined above with a nitrosating agent such as sodium nitrite. The reaction may be performed in a solvent such as aqueous acetic acid at a temperature between 0° C. and 100° C.

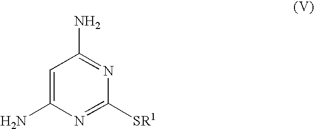

Compounds of formula (V) where R¹ is as defined in formula (I) may be prepared by treating a compound of formula (VI) with a compound of formula R¹X where R¹ is as defined above and X is a leaving group such as bromide in the presence of a base such as potassium tert-butoxide. The reaction may be performed in a solvent such as DMSO at room temperature.

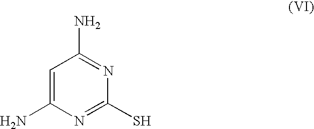

Compounds of formula (IIB) where R¹, R² and R³ are as defined in formula (I) may be prepared by treatment of compounds of formula (VII) where R¹, R² and R³ are as defined above with a reducing agent such as zinc. The reaction may be performed in a solvent such as ethanol at reflux.

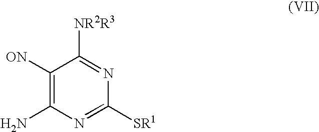

Compounds of formula (VII) where R¹, R² and R³ are as defined in formula (I) may be prepared by treatment of compounds of formula (VIII) where R¹, R² and R³ are as defined above with a nitrosating agent such as sodium nitrite in acetic acid. The reaction may be conveniently carried out at room temperature.

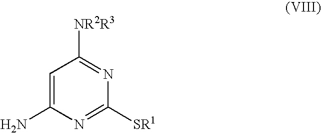

Compounds of formula (VIII) where R¹, R² and R³ are as defined in formula (I) may be prepared by treatment of compounds of formula (IX) where R¹ is as defined in formula (I) and L is a leaving group such as chloro with an amine HNR²R³. The reaction can be carried out in a solvent such as N-methylpyrrolidinone at elevated temperature, for example at between 50° C. and 200° C.

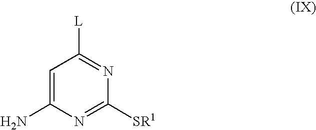

Compounds of formula (IX) where R¹ is as defined in formula (I) and L is a leaving group such as chloro may be prepared by treatment of compounds of formula (IX) where R¹ is as defined in formula (I) and L is hydroxy by treatment with a halogenating agent such as phosphorus oxychloride. The reaction may be carried out in the presence of a base such as 2-picoline at a temperature between 0° C. and 150° C.

Compounds of formula (IX) where R¹ is as defined in formula (I) and L is hydroxy may be prepared from compounds of formula (X) by treatment with a compound of formula R¹X where R¹ is as defined above and X is a leaving group such as bromide. The reaction may be carried out in a solvent such as aqueous DMF using a base such as potassium hydroxide at room temperature.

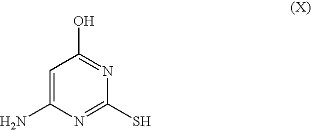

Compounds of formula (VI) and (X) are commercially available.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (I) may involve, at an appropriate stage, the removal of one or more protecting groups. The protection and deprotection of functional groups is fully described in ‘Protective Groups in Organic Chemistry’, edited by J. W. P. McOmie, Plenum Press (1973), and ‘Protective Groups in Organic Synthesis’, 2nd edition, T. W. Greene & P. G. M. Wuts, Wiley-Interscience (1991).

Novel intermediate compounds form a further aspect of the invention. In particular an intermediate of formula (IIA) is provided. Preferably L is bromo; R² is hydrogen; R³ is CH(CH₃)CH₂OH; and R¹is benzyl substituted by two fluorine atoms.

The compounds of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate or p-toluenesulphonate.

The compounds of formula (I) above may be converted to a pharmaceutically acceptable in vivo hydrolysable ester thereof. An in vivo hydrolysable ester of a compound of formula (I) that contains a carboxy or a hydroxy group is, for example a pharmaceutically acceptable ester which is hydrolysed in the human or animal body to produce the parent acid or alcohol. Such s esters can be identified by administering, for example, intravenously to a test animal, the compound under test and subsequently examining the test animal's body fluid.

Suitable pharmaceutically acceptable esters for carboxy include C₁-C₆ alkoxymethyl esters for example methoxymethyl, C₁-C₆ alkanoyloxymethyl esters for example pivaloyloxymethyl, phthalidyl esters, C₃-C₈ cycloalkoxycarbonyloxyC₁-C₆alkyl esters for example 1-cyclohexylcarbonyloxyethyl; 1,3-dioxolen-2-onylmethyl esters for example 5-methyl-1,3-dioxolen-2-onylmethyl; and C₁-C₆ alkoxycarbonyloxyethyl esters for example 1-methoxycarbonyloxyethyl and may be formed at any carboxy group in the compounds of this invention.

Suitable pharmaceutically-acceptable esters for hydroxy include inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2dimethylpropionyloxymethoxy. A selection of in-vivo hydrolysable ester forming groups for hydroxy include C₁-C₁₀ alkanoyl, for example formyl, acetyl; benzoyl; phenylacetyl; substituted benzoyl and phenylacetyl, C₁-C₁₀ alkoxycarbonyl (to give alkyl carbonate esters), for example ethoxycarbonyl; di-(C₁-C₄)alkylcarbamoyl and N-(di-(C₁-C₄)alkylaminoethyl)-N-(C₁-C₄)alkylcarbamoyl (to give carbamates); di-(C₁-C₄)alkylaminoacetyl and carboxyacetyl. Examples of ring substituents on phenylacetyl and benzoyl include aminomethyl, (C₁-C₄)alkylaminomethyl and di-((C₁-C₄)alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O(C₁-C₆)alkyl-CO—, wherein R^{A} is for example, benzyloxy-(C₁-C₄)alkyl, or phenyl). Suitable substituents on a phenyl group in such esters include, for example, 4-(C₁-C₄)piperazio(C₁-C₄)alkyl, piperazino-(C₁-C₄)alkyl and morpholino-(C₁-C₄)alkyl.

The compounds of formula (I) have activity as pharmaceuticals, in particular as modulators of chemokine receptor (especially CXCR2) activity, and may be used in the treatment (therapeutic or prophylactic) of conditions/diseases in human and non-human animals which are exacerbated or caused by excessive or unregulated production of chemokines. Examples of such conditions/diseases include:
(1) (the respiratory tract) obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;
(2) (bone and joints) rheumatoid arthritis, seronegative spondyloarthropathies (including ankylosing spondylitis, psoriatic arthritis and Reiter's disease), Behchet's disease, Sjogren's syndrome and systemic sclerosis;
(3) (skin) psoriasis, atopical dermatitis, contact dermatitis and other eczematous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis;
(4) (gastrointestinal tract) Coeliac disease, proctitis, eosinopilic gastro-enteritis, mastocytosis, Crohn's disease, ulcerative colitis, food-related allergies which have effects remote from the gut, e.g., migraine, rhinitis and eczema;
(5) (central and peripheral nervous system) Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia; polyneuropathies, e.g. Guillain-Barré syndrome, chronic inflammatory demyelinating polyradiculoneuropathy, multifocal motor neuropathy, plexopathies; CNS demyelination, e.g. multiple sclerosis, acute disseminated/haemorrhagic encephalomyelitis, and subacute sclerosing panencephalitis; neuromuscular disorders, e.g. myasthenia gravis and Lambert-Eaton syndrome; spinal diorders, e.g. tropical spastic paraparesis, and stiff-man syndrome: paraneoplastic syndromes, e.g. cerebellar degeneration and encephalomyelitis; CNS trauma; migraine; and stroke;
(6) (other tissues and systemic disease) atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura; post-operative adhesions, and sepsis;
(7) (allograft rejection) acute and chronic following, for example, transplantation of kidney, heart, liver, lung, bone marrow, skin and cornea; and chronic graft versus host disease;
(8) Cancers, especially non-small cell lung cancer (NSCLC), malignant melanoma, prostate cancer and squamous sarcoma, and tumour metastasis, non melanoma skin cancer and chemoprevention of metastases;
(9) Diseases in which angiogenesis is associated with raised CXCR2 chemokine levels (e.g. NSCLC, diabetic retinopathy);
(10) Cystic fibrosis;
(11) Burn wounds & chronic skin ulcers;
(12) Reproductive Diseases (e.g. Disorders of ovulation, menstruation and implantation, Pre-term labour, Endometriosis);
(13) Re-perfusion injury in the heart, brain, peripheral limbs and other organs, inhibition of atherosclerosis.

Thus, the present invention provides a compound of formula (I), or a pharmaceutically-acceptable salt or solvate thereof, as hereinbefore defined for use in therapy.

The present invention also provides a pharmaceutically acceptable in vivo hydrolysable ester of a compound of formula (I), as hereinbefore defined for use in therapy.

Preferably the compounds of the invention are used to treat diseases in which the chemokine receptor belongs to the CXC chemokine receptor subfamily, more preferably the target chemokine receptor is the CXCR2 receptor.

Particular conditions which can be treated with the compounds of the invention are psorisis, rheumatoid arthritis, diseases in which angiogenesis is associated with raised CXCR2 chemokine levels, and respiratory disease such as COPD. It is preferred that the compounds of the invention are used to treat rheumatoid arthritis. The compounds of the invention may also be used to treat COPD.

As a further aspect of the present invention, certain compounds of formula (I) may have utility as antagonists of the CX3CR1 receptor. Such compounds are expected to be particularly useful in the treatment of disorders within the central and peripheral nervous system and other conditions characterized by an activation of microglia and/or infiltration of leukocytes (e.g. stroke/ischemia and head trauma).

In another aspect, the present invention provides the use of a compound of formula (I), or a pharmaceutically acceptable salt or solvate thereof, as hereinbefore defined as a medicament.

In another aspect, the present invention provides the use of a pharmaceutically acceptable in vivo hydrolysable ester of a compound of formula (I), as hereinbefore defined as a medicament.

In a further aspect, the present invention provides the use of a compound of formula (I), or a pharmaceutically acceptable salt or solvate thereof, as hereinbefore defined in the manufacture of a medicament for use in therapy.

The present invention also provides the use of a pharmaceutically acceptable in vivo hydrolysable ester of a compound of formula (I), as hereinbefore defined in the manufacture of a medicament for use in therapy.

In a still further aspect, the present invention provides the use of a compound of formula (I), or a pharmaceutically acceptable salt or solvate thereof, as hereinbefore defined in the manufacture of a medicament for the treatment of human diseases or conditions in which modulation of chemokine receptor activity is beneficial.

The present invention also provides the use of a pharmaceutically acceptable in vivo hydrolysable ester of a compound of formula (I), as hereinbefore defined in the manufacture of a medicament for the treatment of human diseases or conditions in which modulation of chemokine receptor activity is beneficial.

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CXCR2) receptor, which comprises administering to a patient a therapeutically effective amount of a compound of formula (I), or a pharmaceutically acceptable salt or solvate thereof, as hereinbefore defined.

The invention also provides a method of treating a chemokine mediated disease wherein the chemokine binds to a chemokine (especially CXCR2) receptor, which comprises administering to a patient a therapeutically effective amount of a pharmaceutically acceptable in vivo hydrolysable ester of a compound of formula (I), as hereinbefore defined.

The invention also provides a method of treating an inflammatory disease, especially psoriasis, in a patient suffering from, or at risk of, said disease, which comprises administering to the patient a therapeutically effective amount of a compound of formula (I), or a pharmaceutically acceptable salt or solvate thereof, as hereinbefore defined.

The invention further provides a method of treating an inflammatory disease, especially psoriasis, in a patient suffering from, or at risk of, said disease, which comprises administering to the patient a therapeutically effective amount of a pharmaceutically acceptable in vivo hydrolysable ester of a compound of formula (I), as hereinbefore defined

The invention also provides a method of treating an inflammatory disease, especially rheumatoid arthritis, COPD and psoriasis, in a patient suffering from, or at risk of, said disease, which comprises administering to the patient a therapeutically effective amount of a compound of formula (I), or a pharmaceutically acceptable salt, solvate or in vivo hydrolysable ester thereof, as hereinbefore defined. Preferably the method of treating rheumatoid arthritis is provided. Also provided is a method of treating COPD.

For the above-mentioned therapeutic uses the dosage administered will, of course, vary with the compound employed, the mode of administration, the treatment desired and the disorder indicated.

The compounds of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as hereinbefore defined, in association with a pharmaceutically acceptable adjuvant, diluent or carrier.

The present invention further provides a pharmaceutical composition comprising a pharmaceutically acceptable in vivo hydrolysable ester of a compound of formula (I), as hereinbefore defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt or solvate thereof, as hereinbefore defined, with a pharmaceutically acceptable adjuvant, diluent or carrier.

The invention further provides a process for the preparation of a pharmaceutical composition of the invention which comprises mixing a pharmaceutically acceptable in vivo hydrolysable ester of a compound of formula (I), as hereinbefore defined, with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compounds of the invention are administered orally.

The invention will now be further illustrated by reference to the following examples. In the examples the Nuclear Magnetic Resonance (NMR) spectra were measured on a Varian Unity Inova 300 or 400 MHz spectrometer and the Mass Spectrometry (MS) spectra measured on a Finnigan Mat SSQ7000 or Micromass Platform spectrometer or Agilent MSD spectrometer. Where necessary, the reactions were performed under an inert atmosphere of either nitrogen or argon. Chromatography was generally performed using Matrex Silica 60® (35-70 micron) or Prolabo Silica gel 60® (35-70 micron) suitable for flash silica gel chromatography. High pressure liquid chromatography purification was performed using a Waters Micromass LCZ with a Waters 600 pump controller, Waters 2487 detector and Gilson FC024 fraction collector or a Waters Delta Prep 4000 or a Gilson Auto-Purification System. The abbreviations m.p. and DMSO used in the examples stand for melting point and dimethyl sulphoxide respectively.

### EXAMPLES

### Example 1

### 2-[[(2,3-difluorophenyl)methyl]thio]-6-[(2-hydroxyethyl)amino]4-[[(1R)-2hydroxy1-methylethyl]amino]-7(8H)-pteridinone

### a) 2-[[(2,3-Difluorophenyl)methyl]thio]-4,6-pyrimidinediamine

4,6-diamino-2-pyrimidinethiol (7.3 g) was dissolved in DMSO (100 ml) at room temperature under an atmosphere of nitrogen. Potassium tert-butoxide (1M in THF, 48.3 ml) was added followed by 2,3-difluorobenzyl-bromide (10.0 g). The mixture was stirred for 2 hours at room temperature. The reaction mixture was then partitioned between ethyl acetate and ammonium chloride. The organic phase was washed with ammonium chloride (3×) and brine, then dried over magnesium sulphate and evaporated to give the subtitled product as a white solid (12.2 g)

MS: ADCI (+ve) 269 (M+1)

### b) 2-[[(2,3-Difluorophenyl)methyl]thio]-5-nitroso-4,6-pyrimidinediamine

The product of Example 1, step (a) (2.5 g) was dissolved in acetic acid (150 ml) and the solution cooled to 5° C. A solution of sodium nitrite (625 mg) in water (50 ml) was added dropwise resulting in a dark blue colouration. The reaction was stirred at room temperature for 30 minutes during which time a pink solid precipitated from solution. This was isolated by filtration and washed with water, then dried at 50° C. to give the subtitled product as a blue is solid (4.14 g)

MS: ADCI (+ve) 298 (M+1) ¹H NMR: δ (DMSO) 4.44 (s,2H), 7.13-7.54 (m,3H), 8.13 (s,1H), 8.51 (s,1H), 9.10 (s,1H), 10.18 (s,1H).

### c) 4-amino-2-[[(2,3-difluorophenyl)methyl]thio]-7(8H)pteridinone

To a solution of triethyl phosphonoacetate (15.0 g) in tetrahydrofuran (60 ml) cooled in an ice bath was added butyllithium (2.5M in hexanes, 25.6 ml) at a rate such that the internal temperature was maintained below 30° C. To this mixture was then added a solution of the product of Example 1, step (b) (10.0 g) in N,N-dimethylformamide (60 ml). The reaction mixture was heated at reflux for 1 hour, then cooled to room temperature and quenched with acetic acid (6 ml). The solid thus precipitated was isolated by filtration, washed with water, ethanol and diethyl ether, and dried over P₂O₅ at 50° C. to give the sub-titled product as a pale green solid (9.3 g).

MS: ADCI (+ve) 322 (M+1) ¹H NMR: δ (DMSO) 4.18 (s,2H), 7.11-7.58 (m,3H), 7.84 (s,1H), 12.69 (bs,1H)

### d) 4-bromo-2-[[(2,3-difluorophenyl)methyl]thio]-7(8H)-pteridinone

The product of Example 1, step (c) (0.5 g) was suspended in DMSO (10 ml) and bromoform (10 ml) and the mixture was heated to 125° C. Isoamylnitrite (2 ml) was added and the mixture was stirred at 125° C. for 5 minutes before being cooled in an ice bath. Solvent was removed by evaporation under high vacuum and the residue suspended in dichloromethane (100 ml). This suspension was washed with saturated aqueous ammonium chloride (50 ml) and then filtered through a plug of celite. The filtrate was evaporated and purified by column chromatography, eluting with 10% ethyl acetate in dichloromethane to give the subtitled compound as a white solid (0.22 g).

MS: ADCI (+ve) 386 (M+1) ¹H NMR: δ (DMSO) 4.47 (s,2H), 7.13-7.55 (m,3H), 8.14 (s,1H), 13.33 (bs,1H)

### e) 2-[[(2,3-difluorophenyl)methyl]thio]-4-[[(1R)-2-hydroxy-1-methylethyl]amino]-7(8H)-pteridinone

The product of Example 1, step (d) (8.7 g) was dissolved in N-methylpyrrolidinone (40 ml) and Hunigs base (7.9 ml) was added followed by D-alaninol (2.7 ml). The mixture was stirred at 100° C. for 15 mins. The cooled solution was poured onto water, (1 l), and acidified with dilute hydrochloric acid. The solid which separated was collected, washed with water and air dried. Crystallisation from acetonitrile afforded the title compound as a pale yellow solid (7.4 g).

m.p. 215-217° C. MS: APCI (+ve) 380 (M+H, 100%) ¹H NMR: δ (DMSO) 1.14 (d, 3H), 3.48 (m, 2H, 4.31 (m, 1M), 4.45 (dd, 2H) 4.82 (t, 1H) 7.15 (m, 1H), 7.33 (m, 1H), 7.47 (t, 1H), 7.76 (d, 1H), 7.83 (d,1H), 12.70 (s, 1H).

### (f) 6-bromo2-[[(2,3-difluorophenyl)methyl]thio]-4-[[(1R)-2-hydroxy-1-methylethyl]amino]-7(8H)pteridinone

The product of Example 1, step (e) (5.0 g) was suspended in acetonitrile (200 ml) and bromine (1.2 ml) added. The reaction was stirred at room temperature for 2 hours, then evaporated to dryness. The crude product was purified by column chromatography on silica gel, eluting with 2% methanol in dichloromethane to give the subtitled compound as a pale yellow solid (1.7 g).

MS: APCI (+ve) 458/460 (M+H, 100%) ¹H NMR: δ (DMSO) 7.76 (d, 1H), 7.47 (m, 1H), 7.33 (m, 1H, 7.16 (m, 1H), 4.85 (t, 1H), 4.45 (q, 1H), 4.34 (m, 1H), 3.54 (m, 1H), 3.45 (m, 1H), 1.15 (d, 3H).

### (g) 2-[[(2,3-difluorophenyl)methyl]thio]-6-[(2-hydroxyethyl)amino]-4-[[(1R)-2-hydroxy-1-methylethyl]amino]-7(8H)pteridinone

The product of Example 1, step (f) (50 mg), ethanolamine (13 mg) and N,N-diisopropylethylamine (38 ul) were dissolved in N-methylpyrrolidinone (2 ml) and heated to 100° C. for 2 hours. The cooled reaction mixture was diluted with ethyl acetate and washed 5× with saturated aqueous ammonium chloride. The organic phase was dried over magnesium sulphate, filtered and evaporated. The crude product was purified by column chromatography on silica gel, eluting with 10% methanol in dichloromethane, followed by trituation with methanol to give the titled compound as a white solid (10 mg).

m.p. 197-199° C. MS: APCI (+ve) 439 (M+H, 100%) ¹H NMR: δ (DMSO) 12.45 (br s, 1H), 7.45 (m, 1H), 7.29 (m, 2H), 7.14 (m, 1H), 6.66 (d, 1H), 4.87 (t, 1H), 4.77 (t, 1H), 4.40 (s, 2H), 4.22 (m, 1H), 3.59 (m, 2H), 3.49 (m, 4H), 1.16 (d, 3H).

### Example 2

### 2-[[(2,3-difluorophenyl)methyl]thio]-4-[[(1R)-2-hydroxy-1-methylethyl]amino]-6-[(phenylmethyl)amino]-7(8H)-pteridinone

The product of Example 1, step (f) (50 mg), benzylamine (35 mg) and N,N-diisopropylethylamine (38 ul) were dissolved in N-methylpyrrolidinone (2 ml) and heated to 100° C. for 2 hours. The cooled reaction mixture was diluted with ethyl acetate and washed 5× with saturated aqueous ammonium chloride. The organic phase was dried over magnesium sulphate, filtered and evaporated. The crude product was purified by column chromatography on silica gel, eluting with 2% methanol in dichloromethane to give the titled compound as a pale yellow solid (24 mg).

m.p. 80-100° C. MS: APCI (+ve) 485 (M+H, 100%) ¹H NMR: δ (DMSO) 9.10 (br s, 1H), 7.35 (m, 6H), 7.02 (m, 2H), 6.57 (br t, 1H), 6.07 (br, d, 1H), 4.62 (m, 2H), 4.38 (s, 2H), 4.33 (m, 1H), 3.76 (m, 1H), 3.64 (m, 1H), 2.62 (m, 1H), 1.28 (d, 3H).

### Example 3

### 2-[[(2,3-Difluorophenyl)methyl]thio-4-[[(1R)-2-hydroxy-1-methylethyl]amino]-6,7-pteridinedione

### (a) 6-amino-2-[[(2,3-difluorophenyl)methyl]thio]-4-pyrimidinol,

To a stirred suspension of 4-amino-6-hydroxy-2-mercaptopyrimidine monohydrate (50 g) in DMP (600 ml) was added potassium hydroxide (20.2 g), water (100 ml) and 2,3-difluorobenzyl bromide (64.2 g). The reaction was stirred at room temperature for 30 min. The mixture was poured into water (4 l) and the resulting precipitate isolated by filtration, washing with isopropanol, to afford the subtitled compound as a white solid (73.7 g).

m.p. 218-221° C. MS: APCI+ve 270 (M+H, 100%) ¹H NMR: δ (DMSO) 7.37 (1H, m), 7.35 (1H, m), 7.16 (1H, m), 6.57 (3H, m), 4.99 (1H, br s), 4.39 (2H, s).

### (b) 6-chloro-2-[[(2,3-difluorophenyl)methyl]thio]-4-pyrimidinamine

The product of Example 3, step (a) (73 g) was added to a solution of 2-picoline (40 ml) in POCl₃ (300 ml) and the mixture refluxed for 24 hr. The reaction mixture was concentrated in vacuo to half its original volume and poured onto ice and then neutralized with ammonia, forming a brown solid. This mixture was refluxed for 1 hr, and the precipitate isolated by filtration, washing with water. The crude product was purified by silica gel chromatography, eluting with dichloromethane to afford the subtitled compound as a white solid (31.7 g).

MS: APCI (+ve) 288/290 (M+H, 100%) ¹H NMR: δ (DMSO) 7.33 (5H, m), 7.12 (1H, m), 4.36 (2H, s).

### (c) (2R)-2-[[6-amino-2-[[(2,3difluorophenyl)methyl]thio]-4-pyrimidinyl]amino]-1-propanol

A solution of the product from Example 3, step (b) (22 g), N,N-diisopropylethylamine (110 ml) and D-alaninol (23 g) in NMP (150 ml) was heated at 160° C. for 48 h. The reaction mixture was allowed to cool to room temperature and poured into aqueous ammonium chloride (1.2 l). The resultant white precipitate was purified by silica gel chromatography, eluting first with 3:2 DCM:ethyl acetate and then 3:1 DCM:methanol, giving the subtitled product as a pink solid (22.6 g).

MS: APCI (+ve) 327 (M+H, 100%) ¹H NMR: δ (DMSO) 7.38 (1H, m), 7.29 (1H, m), 7.10 (1H, m), 6.40 (1H, d), 6.13 (2H, bs), 5.15 (1H, s), 4.66 (1H, t), 4.31 (2H, t), 4.02 (1H, bs), 3.39 (1H, m), 3.25 (1H, m), 1.05 (3H, d).

### (d) (R)-2-[[6-amino-2-[[(2,3-difluorophenyl)methyl]thio]-5-nitroso-4-pyrimidinyl]amino]-1-propanol

To a stirred solution of the product from Example 3, step (c) (22 g) in acetic acid (300 ml) at room temperature was added a solution of sodium nitrite (4.8 g) in water (30 ml). The reaction was stirred at 0° C. for 30 min, and the resultant purple precipitate isolated by filtration, washing with water, to give the subtitled compound as a dark blue solid (37 g, not completely dry).

MS: APCI (+ve) 356 (M+H, 100%)

### (e) (2R)-2-[[5,6-diamino-2-[[(2,3-difluorophenyl)methyl]thio]-4-pyrimidinyl]amino]-1-propanol

To a solution of acetic acid (10 ml) in boiling ethanol (300 ml) was added zinc dust (15 g) and the product of Example 3, step (d) (10 g). The reaction was heated at reflux for 10 mins, cooled, filtered through celite and the filtrate evaporated. The crude product was triturated with water, filtered and dried in vacuo to to give the subtitled product as a cream solid (9.3 g).

MS: APCI (+ve) 342 (M+H, 100%) ¹H NMR: δ (DMSO) 7.34 (1H, m), 7.27 (1H, m), 7.12 (1H, m), 5.72 (2H, b s), 5.58 (1H, d), 4.65 (1H, t), 4.30 (2H, d), 4.04 (1H, m), 3.54 (2H, bs), 3.44 (1H, m), 3.29 (1H, m), 1.09 (3H, d).

### (f) 2-[[(2,3-Difluorophenyl)methyl]thio-4-[[(1R)-2-hydroxy-1-methylethyl]amino]-6,7-pteridinedione

The product of Example 3, step (e) (0.30 g) and diethyloxalate were heated at 160° C. for 30 mins. The mixture was concentrated in vacuo. Purification by flash chromatography over silica using dichloromethane/methanol (9:1) as eluant afforded the title compound (0.045 g).

m.p. 243-246° C. MS: APCI 396 (M+H, 100%) ¹H NMR: δ (DMSO) 1.12 (d, 3H), 3.43 (m, 2H), 4.14 (m, 1H), 4.38 (q, 2H), 6.79 (d, 1H), 7.13 (m, 1H), 7.30 (m, 1H), 7.45 (t, 1H).

### Example 4

### 6-amino-2-[[(2,3-difluorophenyl)methyl]thio]-4-[[(1R)-2-hydroxy-1-methylethyl]amino]-7(8H)-pteridinone

To a solution of the product from Example 3, step (e) (190 mg) in ethanol (30 ml) was added iminomethoxy-acetic acid, methyl ester hydrochloride (85 mg) (J. Chem. Soc., Perkin 1, 1999, 1783-93) followed by N,N-diisopropylethylamine (70 ul) and the mixture heated under reflux is for 24 hours. The mixture was evaporated to dryness and purified by silica chromatography (ethyl acetate) to give the title compound (55 mg).

MS APCI+ve 395 (M+H, 100%) ¹H NMR: δ (DMSO) 12.45 (bs, 1H), 7.45 (t, 1H), 7.31 (m, 1H), 7.27 (m, 1H), 7.06 (bst, 2H), 6.49 (d,1H), 4.91 (t, 1H), 4.36 (ab, 2H), 4.17 (m, 1H), 3.47 (t, 2H), 1.13 (d, 3H).

### Example 5

### 2-[(2,3-Difluorophenyl)methyl)thio]-4-[[(1R)-2-hydroxy-1-methylethyl]amino]-6-(1H-imidazol-1-yl)-7(8H)-pteridinone

The product of Example 1, step (f) (250 mg), imidazole (445 mg) and potassium tert-butoxide (5.5 ml, 1M solution in THF) were dissolved in DMSO (10 ml) and the mixture heated to 100° C. for 1 hour. The cooled reaction mixture was diluted with ethyl acetate and washed 2× with saturated aqueous ammonium chloride and 2× with water. The organic phase was dried over magnesium sulphate, filtered and evaporated to give a yellow solid, which was recrystallised twice from acetonitrile/methanol to give the title compound (30 mg).

MS APCI+ve 446 (M+H, 100%) ¹H NMR: δ (DMSO) 13.19 (s, 1H), 8.95 (s, 1H), 8.38 (s, 1H), 7.71 (d, 1H), 7.49 (m, 1H), 7.34 (m, 1H), 7.16 (m, 1H), 7.11 (s,1H), 4.85 (t, 1H), 4.46 (ab, 2H), 4.30 (m, 1H), 3.50 (m, 2H), 1.19 (d, 3H).

### Example 6

### 2-[[2,3-Difluorophenyl)methyl)thio]-4-[[(1R)-2-hydroxy-1-methylethyl]amino]-6-(1-methyl-1H-imidazol-2-yl)thio]-7(8H)-pteridinone

The product of Example 1, step (f) (250 mg), 1-methyl-1H-imidazole-2-thiol (375 mg) and butyllithium (0.6 ml, 2.5M solution in hexanes) were dissolved in N-methylpyrrolidinone (10 ml) and the mixture heated to 100° C. for 2 hours. The cooled reaction mixture was diluted with ethyl acetate and washed 4× with saturated aqueous ammonium chloride. The organic phase was dried over magnesium sulphate, filtered and evaporated. The crude product was purified by silica chromatography (20:1 dichloromethane:methanol) to give the title compound (95 mg).

m.p. 216-217° C. MS APCI+ve 492 (M+H, 100%) ¹H NMR: δ (DMSO) 13.00 (s, 1H), 7.52 (s, 1H), 7.42 (t, 1H), 7.30 (m, 1H), 7.13 (m, 2H), 5.97 (d, 1H), 4.90 (br s, 1H), 4.42 (s, 2H), 4.05 (m, 1H), 3.59 (s, 3H), 3.38 (m, 2H), 1.06 (d, 3H).

### Example 7

### 2-[[2,3-Difluorophenyl)methyl)thio]-4-[[(1R)-2-hydroxy-1-methylethyl]amino]-6-methoxy-7(8H)-pteridinone

The product of Example 1, step (f) (250 mg) was dissolved in methanol (10 ml) and butyllithium (0.6 ml, 2.5M solution in hexanes) was added. The mixture was heated at reflux for 48 hours. The cooled reaction mixture was evaporated and purified by silica chromatography (20:1 dichloromethane:methanol) followed by recrystallisation from methanol to give the title compound (15 mg).

MS APCI+ve 410 (M+H, 100%) ¹H NMR: δ (DMSO) 7.46 (t, 1H), 7.32 (q, 1H), 7.13 (m, 1H), 6.96 (br d, 1H), 4.86 (t, 1H), 4.42 (ab, 2H), 4.28 (m, 1H), 3.97 (s, 3H), 3.47 (m, 2H), 1.16 (d, 3H).

### Examples 8 to 36

Examples 8 to 36 were prepared by the method of Example 1 step (g) by reaction of the product of Example 1, step (f) with the appropriate amine or thiol. The product purified by either (a) column chromatography on silica gel, eluting with 10% methanol in dichloromethane, followed by trituration with methanol or (b) reverse phase chromatography using a Waters Xterra column with acetonitrile and 0.2% 0.880 NH₄OH solution as buffer to give the products as a solid as shown in table 1.

TABLE 1

| Example | | |
|---|---|---|
| Number | Compound Name | MS: APCI (+ve) |
| 8 | 2-[[2,3-difluorophenyl)metbyl]thio]-4-[[(1R)-2-hydroxy- | 486 |
| | 1-methylethyl]amino]-6-[(3-pyridinylmethyl)amino]- | (M + H, |
| | 7(8H)-pteridinone | 100%) |
| 9 | 2-[[(2,3-difluorophenyl)methyl]thio]-4-[[(1R)-2-hydroxy- | 489 |
| | 1-methylethyl]amino]-6-[[(5-methyl-2- | (M + H, |
| | furanyl)methyl]amino]-7(8H)-pteridinone | 100%) |
| 10 | 2-[[2,3-difluorophenyl)methyl]thio]-6-[(3R,5S)-3,5- | 492 |
| | dimethyl-1-piperazinyl]-4-[[(1R)-2-hydroxy-1- | (M + H, |
| | methylethyl]amino]-7(8H)-pteridinone | 100%) |
| 11 | 2-[[(2,3-difluorophenyl)methyl]thio]-4-[[(1R)-2-hydroxy- | 446 |
| | 1-methylethyl]amino]-6-[methyl[(3-methyl-5- | (M + H, |
| | isoxazolyl)methyl]amino]-7(8H)-pteridinone | 100%) |
| 12 | 2-[[(2,3-difluorophenyl)methyl]thio]-4-[[(1R)-2-hydroxy- | 516 |
| | 1-methylethyl]amino]-6-[[2-(2- | (M + H, |
| | pyrimidinylamino)ethyl]amino]-7(8H)-pteridinone | 100%) |
| 13 | 2-[[(2,3-difluorophenyl)methyl]thio]-4-[[(1R)-2-hydroxy- | 465 |
| | 1-methylethyl]amino]-6-(4-morpholinyl)-7(8H)-pteridinone | (M + H, |
| | | 100%) |
| 14 | 2-[[(2,3-difluorophenyl)methyl]thio]-4-[[(1R)-2-hydroxy- | 508 |
| | 1-methylethyl]amino]-6-[[2-(4-morpholinyl)ethyl]amino]- | (M + H, |
| | 7(8H)-pteridinone | 100%) |
| 15 | 2-[[(2,3-difluorophenyl)methyl]thio]-4-[[(1R)-2-hydroxy- | 453 |
| | 1-methylethyl]amino]-6-[(2-methoxyethyl)amino]-7(8H)- | (M + H, |
| | pteridinone | 100%) |
| 16 | 2-[[(2,3-difluorophenyl)methyl]thio]-6-[(2- | 475 |
| | furanylmethyl)amino]-4-[[(1R)-2-hydroxy-1- | (M + H, |
| | methylethyl]amino]-7(8H)-pteridinone | 100%) |
| 17 | 6-(1-azetidinyl)-2-[[(2,3-difluorophenyl)methyl]thio]-4- | 435 |
| | [[(1R)-2-hydroxy-1-methylethyl]amino]-7(8H)-pteridinone | (M + H, |
| | | 100%) |
| 18 | 2-[[(2,3-difluorophenyl)methyl]thio]-4-[[(1R)-2-hydroxy- | 501 |
| | 1-methylethyl]amino]-6-[[(5- | (M + H, |
| | methylpyrazinyl)methyl]amino]-7(8H)-pteridinone | 100%) |
| 19 | 2-[[(2,3-difluorophenyl)methyl]thio]-6-[[2-(2- | 489 |
| | furanyl)ethyl]amino]-4-[[(1R)-2-hydroxy-1- | (M + H, |
| | methylethyl]amino]-7(8H)-pteridinone | 100%) |
| 20 | 2-[[(2,3-difluorophenyl)methyl]thio]-4-[[(1R)-2-hydroxy- | 522 |
| | 1-methylethyl]amino]-6-[[3-(4-morpholinyl)propyl]amino]- | (M + H, |
| | 7(8H)-pteridinone | 100%) |
| 21 | 2-[[(2,3-difluorophenyl)methyl]thio]-4-[[(1R)-2-hydroxy- | 490 |
| | 1-methylethyl]amino]-6-[[(3-methyl-5- | (M + H, |
| | isoxazolyl)methyl]amino]-7(8H)-pteridinone | 100%) |
| 22 | 2-[[(2,3-difluorophenyl)methyl]thio]-4-[[(1R)-2-hydroxy- | 465 |
| | 1-methylethyl]amino]-6-[(3S)-3-hydroxy-1-pyrrolidinyl]- | (M + H, |
| | 7(8H)-pteridinone | 100%) |
| 23 | 2-[[(2,3-difluorophenyl)methyl]thio]-6-[(2- | 492 |
| | furanylmethyl)thio]-4-[[(1R)-2-hydroxy-1- | (M + H, |
| | methylethyl]amino]-7(8H)-pteridinone | 100%) |
| 24 | 2-[[(2,3-difluorophenyl)methyl]thio]-4-[[(1R)-2-hydroxy- | 453 |
| | 1-methylethyl]amino]-6-[(2-hydroxypropyl)amino]-7(8H)- | (M + H, |
| | pteridinone | 100%) |
| 25 | 2-[[(2,3-difluorophenyl)methyl]thio]-6-[[2- | 483 |
| | (dimethylamino)ethyl]thio]-4-[[(1R)-2-hydroxy-1- | (M + H, |
| | methylethyl]amino]-7(8H)-pteridinone | 100%) |
| 26 | 2-[[(2,3-difluorophenyl)methyl]thio]-4-[[(1R)-2-hydroxy- | 453 |
| | 1-methylethyl]amino]-6-[[(2S)-2-hydroxypropyl]amino]- | (M + H, |
| | 7(8H)-pteridinone | 100%) |
| 27 | 2-[[(2,3-difluorophenyl)methyl]thio]-4-[[(1R)-2-hydroxy- | 453 |
| | 1-methylethyl]amino]-6-[(3-hydroxypropyl)amino]-7(8H)- | (M + H, |
| | pteridinone | 100%) |
| 28 | 2-[[(2,3-difluorophenyl)methyl]thio]-6-[(2- | 453 |
| | hydroxyethyl)methylamino]-4-[[(1R)-2-hydroxy-1- | (M + H, |
| | methylethyl]amino]-7(8H)-pteridinone | 100%) |
| 29 | 2-[[(2,3-difluorophenyl)methyl]thio]-4-[[(1R)-2-hydroxy- | 509 |
| | 1-methylethyl]amino]-6-[(5-hydroxy-4-methyl-4H-1,2,4- | (M + H, |
| | triazol-3-yl)thio]-7(8H)-pteridinone | 100%) |
| 30 | 2-[[(2,3-difluorophenyl)methyl]thio]-6-[(4- | 493 |
| | hydroxycyclohexyl)amino]-4-[[(1R)-2-hydroxy-1- | (M + H, |
| | methylethyl]amino]-7(8H)-pteridinone | 100%) |
| 31 | 2-[[(2,3-difluorophenyl)methyl]thio]-4-[[(1R)-2-hydroxy- | 496 |
| | 1-methylethyl]amino]-6-(1,3,4-thiadiazol-2-ylthio)-7(8H)- | (M + H, |
| | pteridinone | 100%) |
| 32 | 2-[[(2,3-difluorophenyl)methyl]thio]-6-[[(1S,4R)-4- | 477 |
| | hydroxy-2-cyclopenten-1-yl]amino]-4-[[(1R)-2-hydroxy-1- | (M + H, |
| | methylethyl]amino]-7(8H)-pteridinone | 100%) |
| 33 | 2-[[(2,3-difluorophenyl)methyl]thio]-4-[[(1R)-2-hydroxy- | 465 |
| | 1-methylethyl]amino]-6-[(3R)-3-hydroxy-1-pyrrolidinyl]- | (M + H, |
| | 7(8H)-pteridinone | 100%) |
| 34 | 2-[[(2,3-difluorophenyl)methyl]thio]-6-(3-hydroxy-3- | 465 |
| | methyl-1-azetidinyl)-4-[[(1R)-2-hydroxy-1- | (M + H, |
| | methylethyl]amino]-7(8H)-pteridinone | 100%) |
| 35 | 6-[(3S)-3-amino-1-pyrrolidinyl]-2-[[(2,3- | 464 |
| | difluorophenyl)methyl]thio]-4-[[(1R)-2-hydroxy-1- | (M + H, |
| | methylethyl]amino]-7(8H)-pteridinone | 100%) |
| 36 | 6-[(2-aminoethyl)thio]-2-[[(2,3- | 453 |
| | difluorophenyl)methyl]thio]-4-[[(1R)-2-hydroxy-1- | (M + H, |
| | methylethyl]amino]-7(8H)-pteridinone | 100%) |

Pharmacological Data
Ligand Binding Assay

[¹²⁵I]IL-8(human, recombinant) was purchased from Amersham, U.K. with a specific activity of 2,000 Ci/mmol. All other chemicals were of analytical grade. High levels of hrCXCR2 were expressed in HEK 293 cells (human embryo kidney 293 cells ECACC No. 85120602) (Lee et al. (1992) J. Biol. Chem. 267 pp16283-16291). hrCXCR2 cDNA was amplified and cloned from human neutrophil mRNA. The DNA was cloned into PCRScript (Stratagene) and clones were identified using DNA. The coding sequence was subcloned into the eukaryotic expression vector RcCMV (Invitrogen). Plasmid DNA was prepared using Quiagen Megaprep 2500 and transfected into HEK 293 cells using Lipofectamine reagent (Gibco BRL). Cells of the highest expressing clone were harvested in phosphate-buffered saline containing 0.2% (w/v) ethylenediaminetetraacetic acid (EDTA) and centrifuged (200 g, 5 min.). The cell pellet was resuspended in ice cold homogenisation buffer [10 mM HEPES (pH 7.4), 1 mM dithiothreitol, 1 mM EDTA and a panel of protease inhibitors (1 mM phenyl methyl sulphonyl fluoride, 2 μg/ml soybean trypsin inhibitor, 3 mM benzamidine, 0.5 μg/ml leupeptin and 100 μg/ml bacitracin)] and the cells left to swell for 10 minutes. The cell preparation was disrupted using a hand held glass mortar/PTFE pestle homogeniser and cell membranes harvested by centrifugation (45 minutes, 100,000 g, 4° C.). The membrane preparation was stored at −70° C. in homogenisation buffer supplemented with Tyrode's salt solution (137 mM NaCl, 2.7 mM KCl, 0.4 mM NaH₂PO₄), 0.1% (w/v) gelatin and 10% (v/v) glycerol.

All assays were performed in a 96-well MultiScreen 0.45 μm filtration plates (Mllipore, U.K.). Each assay contained ˜50 pM [125I]IL-8 and membranes (equivalent to ˜200,000 cells) in assay buffer [Tyrode's salt solution supplemented with 10 mM HEPES (pH 7.4), 1.8 mM CaCl₂, 1 mM MgCl₂, 0.125 mg/ml bacitracin and 0.1% (w/v) gelatin]. In addition, a compound of formula (I) according to the Examples was predissolved in DMSO and added to reach a final concentration of 1% (v/v) DMSO. The assay was initiated with the addition of membranes and after 1.5 hours at room temperature the membranes were harvested by filtration using a Millipore MultiScreen vacuum manifold and washed twice with assay buffer (without bacitracin). The backing plate was removed from the MultiScreen plate assembly, the filters dried at room temperature, punched out and then counted on a Cobra γ-counter.

The compounds of formula (I) according to the Examples were found to have IC₅₀ values of less than (<) 10 μM.

Intracellular Calcium Mobilisation Assay

Human neutrophils were prepared from EDTA-treated peripheral blood, as previously described (Baly et al. (1997) Methods in Enzymology 287 pp70-72), in storage buffer ∂Tyrode's salt solution (137 mM NaCl, 2.7 mM KCl, 0.4 mM NaH₂PO₄) supplemented with 5.7 mM glucose and 10 mM HEPES (pH 7.4)].

The chemokine GROα (human, recombinant) was purchased from R&D Systems (Abingdon, U.K.). All other chemicals were of analytical grade. Changes in intracellular free calcium were measured fluorometrically by loading neutrophils with the calcium sensitive fluorescent dye, fluo-3, as described previously (Merritt et al. (1990) Biochem. J. 269, pp513-519). Cells were loaded for 1 hour at 37° C. in loading buffer (storage buffer with 0.1% (w/v) gelatin) containing 5 μM fluo-3 AM ester, washed with loading buffer and then resuspended in Tyrode's salt solution supplemented with 5.7 mM glucose, 0.1% (w/v) bovine serum albumin (BSA), 1.8 mM CaCl₂ and 1 mM MgCl₂. The cells were pipetted into black walled, clear bottom, 96 well micro plates (Costar, Boston, U.S.A.) and centrifuged (200 g, 5 minutes, room temperature).

A compound of formula (I) according to the Examples was pre-dissolved in DMSO and added to a final concentration of 0.1% (v/v) DMSO. Assays were initiated by the addition of an A₅₀concentration of GROα and the transient increase in fluo-3 fluorescence (λ_{Ex}=490 nm and λ_{Em}520 nm) monitored using a FLIPR (Fluorometric Imaging Plate Reader, Molecular Devices, Sunnyvale, U.S.A.).

The compounds of formula (I) according to the Examples were tested and found to be antagonists of the CXCR2 receptor in human neutrophils.

1. A compound of formula (I) or a pharmaceutically acceptable salt, solvate or in vivo hydrolysable ester thereof: 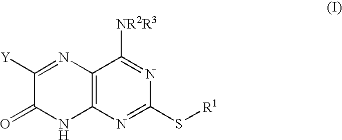
in which:
R¹ represents a C₃-C₇ carbocyclic, C₁-C₈ alkyl, C₂-C₆ alkenyl or C₂-C₆ alkynyl group, each of which may be optionally substituted by one or more substituent groups independently selected from halogen atoms, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, an aryl or heteroaryl group, which last two may themselves be optionally substituted by one or more substituents independently selected from halogen atoms, cyano, nitro, —OR⁴, —NR⁵R⁶, —CONR⁵R⁶, —COOR⁷, —NR⁸COR⁹, —SR¹⁰, —SO₂R¹⁰, —SO₂NR⁵R⁶, —NR⁸SO₂R⁹, C₁-C₆ alkyl or trifluoromethyl groups;
R² and R³ each independently represent a hydrogen atom, or a C₃-C₇ carbocyclic, C₁-C₈ alkyl, C₂-C₆ alkenyl or C₂-C₆ alkynyl group, the latter four groups may be optionally substituted by one or more substituent groups independently selected from:
(a) halogen atoms, —OR⁴, —NR⁵R⁶—CONR⁵R⁶, —COOR⁷, —NR⁸COR⁹, —SR¹⁰, —SO₂R¹⁰, —SO₂NR⁵R⁶, —NR⁸SO₂R⁹;
(b) a 3-8 membered ring optionally containing one or more atoms selected from O, S, NR⁸ and itself optionally substituted by C₁-C₃-alkyl or halogen; or
(c) an aryl group or heteroaryl group each of which may be optionally substituted by one or more substituents independently selected from halogen atoms, cyano, nitro, —OR⁴, —NR⁵R⁶, -—CONR⁵R⁶, —NR⁸COR⁹, —SO₂NR⁵R⁶, —NR⁸SO₂R⁹, C₁-C₆ alkyl and trifluoromethyl groups;
R⁴ represents hydrogen or a C₁-C₆ alkyl group which may be optionally substituted by one or more substituent groups independently selected from halogen atoms, —OR¹¹, —NR⁵R⁶, or an aryl group or heteroaryl group either of which may be optionally substituted by one or more substituents independently selected from halogen atoms, cyano, nitro, —OR¹¹, —NR⁵R⁶, —CONR⁵R⁶, —NR⁸COR⁹, —SO₂NR⁵R⁶, —NR⁸SO₂R⁹, C₁-C₆ alkyl and trifluoromethyl groups; or R⁴ represents a halogen atom, —OR¹¹, —NR⁵R⁶, or an aryl group or heteroaryl group either of which may be optionally substituted by one or more substituents independently selected from halogen atoms, cyano, nitro, —OR¹¹, —NR⁵R⁶, —CONR⁵R⁶, —NR⁸COR⁹, —SO₂NR⁵R⁶, —NR⁸SO₂R⁹, C₁-C₆ alkyl and trifluoromethyl groups;
R⁵ and R⁶ independently represent a hydrogen atom or a C₁-C₆ alkyl or phenyl group or heteroaryl group the latter three of which may be optionally substituted by one or more substituent groups independently selected from halogen atoms, phenyl, —OR¹⁴ and —NR¹⁵R¹⁶, —CONR¹⁵R¹⁶, —NR¹⁵COR¹⁶, —SONR¹⁵R¹⁶, NR¹⁵SO₂R¹⁶
or
R⁵ and R⁶ together with the nitrogen atom to which they are attached form a 4- to 7-membered saturated heterocyclic ring system optionally containing a further heteroatom selected from oxygen and nitrogen atoms, which ring system may be optionally substituted by one or more substituent groups independently selected from phenyl, —OR¹⁴, —COOR¹⁴, —NR¹⁵R¹⁶, —CONR¹⁵R¹⁶, —NR¹⁵COR¹⁶, —SONR¹⁵R¹⁶, —NR¹⁵SO₂R¹⁶ or C₁-C₆ alkyl, itself optionally substituted by one or more substituents independently selected from halogen atoms and —NR¹⁵R¹⁶ and —OR¹⁷ groups;R¹⁰ represents a C₁-C₆-alkyl or a phenyl group, either of which may be optionally substituted by one or more substituent groups independently selected from halogen atoms, phenyl, —OR¹⁷ and —NR¹⁵R¹⁶,
Y is NR²⁰R²¹, OR⁴, SR⁴, a heteroaryl group or NR⁵R⁶ where R⁵ and R⁶ together with the nitrogen atom to which they are attached form a 4- to 7-membered saturated heterocyclic ring system optionally containing a further heteroatom selected from oxygen and nitrogen atoms, which ring system may be optionally substituted by one or more substituent groups independently selected from phenyl, —OR¹⁴, —COOR¹⁴, —NR¹⁵R¹⁶, —CONR¹⁵R¹⁶, —NR¹⁵COR¹⁶, —SONR¹⁵R¹⁶, NR¹⁵SO₂R¹⁶ or C₁-C₆ alkyl, itself optionally substituted by one or more substituents independently selected from halogen atoms and -NR¹⁵R¹⁶ and —OR¹⁷ groups;
each of R⁷, R⁸, R⁹, R¹¹, R¹⁵, R¹⁶and R¹⁷ independently represents a hydrogen atom or a C₁-C₆, alkyl, or a phenyl group; and
R²⁰ and R²¹ are defined as for R² and R³.

2. A compound according to claim 1, wherein R¹ represents an optionally substituted benzyl group.

3. A compound according to claim 2, wherein R¹ represents benzyl substituted by two halogen atoms.

4. A compound according to claim 1, wherein one of R² and R³ is hydrogen and the other is C₃-C₄ alkyl substituted by one or more hydroxy groups.

5. A compound according to claim 1 wherein one of R² and R³ is hydrogen and the other is CH(CH₃)CH₂OH, CH(Et)CH₂OH, C(CH₃)₂CH₂OH or CH(CH₂OH)₂.

6. A compound according to claim 1 wherein one of R² and R³ is hydrogen and the other is CH(CH₃)CH₂OH.

7. A compound according to claim 6 in the form of the (R) isomer.

8. A compound according to claim 1 wherein Y is —NR²⁰R²¹, —OR⁴, —SR⁴, a heteroaryl group or —NR⁵R⁶ where R⁵ and R⁶ together with the nitrogen atom to which they are attached form a 4- to 7-membered saturated heterocyclic ring system optionally containing a further heteroatom selected from oxygen and nitrogen atoms, which ring system may be optionally substituted by one or more substituent groups independently selected from —OH, —NH₂ or C₁-C₄ alkyl.

9. A compound according to claim 1 selected from:
2-[[(2,3-difluorophenyl)methyl]thio]-6-[(2-hydroxyethyl)amino]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[(phenylmethyl)amino]-7(8H)-pteridinone;
2-[[(2,3-Difluorophenyl)methyl]thio-4-[[(1R)-2-hydroxy-1-methylethyl]amino]-6,7-pteridinedione;
6-amino-2-[[(2,3-difluorophenyl)methyl]thio]-4-[[(1R)-2-hydroxy-1-methylethyl]amino]-7(8H)-pteridinone;
2-[[2,3-difluorophenyl)methyl)thio]-4-[[(1R)-2-hydroxy-1-methylethyl]amino]-6-(1H-imidazol-1-yl)-7(8H)-pteridinone;
2-[[2,3-difluorophenyl)methyl)thio]-4-[[(1R)-2-hydroxy-1-methylethyl]amino]-6-[(1-methyl-1H-imidazol-2-yl)thio]-7(8H)-pteridinone;
2-[[2,3-difluorophenyl)methyl)thio]-4-[[(1R)-2-hydroxy-1-methylethyl]amino]-6-methoxy-7(8H)-pteridinone;
2-[[(2,3-difluorophenyl)methyl]thio]-4-[[(1R)-2-hydroxy-1-methylethyl]amino]-6-[(3-pyridinylmethyl)amino]-7(8H)-pteridinone;
2-[[(2,3-difluorophenyl)methyl]thio]-4-[[(1R)-2-hydroxy-1-methylethyl]amino]-6-[[(5-methyl-2-furanyl)methyl]amino]-7(8H)-pteridinone;
2-[[(2,3-difluorophenyl)methyl]thio]-6-[(3R,5S)-3,5-dimethyl-1-piperazinyl]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[methyl[(3-methyl-5-isoxazolyl)methyl]amino]-7(8H)-pteridinone;
2-[[(2,3-difluorophenyl)methyl]thio]-4-[[(1R)-2-hydroxy-1-methylethyl]amino]-6-[[2-(2-pyrimidinylamino)ethyl]amino]-7(8H)-pteridinone;
2-[[(2,3-difluorophenyl)methyl]thio]-4-[[(1R)-2-hydroxy-1-methylethyl]amino]-6-(4-morpholinyl)-7(8H)-pteridinone;
2-[[(2,3-difluorophenyl)methyl]thio]-4-[[(1R)-2-hydroxy-1-methylethyl]amino]-6-[[2-(4-morpholinyl)ethyl]amino]-7(8H)-pteridinone;
2-[[(2,3-difluorophenyl)methyl]thio]-4-[[(1R)-2-hydroxy-1-methylethyl]amino]-6-[(2-methoxyethyl)amino]-7(8H)-pteridinone;
2-[[(2,3-difluorophenyl)methyl]thio]-6-[(2-furanylmethyl)amino]-4-[[(1R)-2-hydroxy-1-methylethyl]amino]-7(8H)-pteridinone;
6-(1-azetidinyl)-2-[[(2,3-difluorophenyl)methyl]thio]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[[5-methylpyrazinyl)methyl]amino]-7(8H)-pteridinone;
2-[[(2,3-difluorophenyl)methyl]thio]-6-[[2-(2-furanyl)ethyl]amino]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[[3-(4-morpholinyl)propyl]amino]-7(8H)-pteridinone;
2-[[(2,3-difluorophenyl)methyl]thio]-4-[[(1R)-2-hydroxy-1-methylethyl]amino]-6-[[(3-methyl-5-isoxazolyl)methyl]amino]-7(8H)-pteridinone;
2-[[(2,3-difluorophenyl)methyl]thio]-4-[[(1R)-2-hydroxy-1-methylethyl]amino]-6-[(3S)-3-hydroxy-1-pyrrolidinyl]-7(8H)-pteridinone;
2-[[(2,3-difluorophenyl)methyl]thio]-6-[(2-furanylmethyl)thio]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[(2-hydroxypropyl)amino]-7(8H)-pteridinone;
2-[[(2,3-difluorophenyl)methyl]thio]-6-[[2-(dimethylamino)ethyl]thio]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[[(2S)-2-hydroxypropyl]amino]-7(8H)-pteridinone;
2-[[(2,3-difluorophenyl)methyl]thio]-4-[[(1R)-2-hydroxy-1-methylethyl]amino]-6-[(3-hydroxypropyl)amino]-7(8H)-pteridinone;
2-[[(2,3-difluorophenyl)methyl]thio]-6-[(2-hydroxyethyl)methylamino]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[(5-hydroxy-4-methyl-4H-1,2,4-triazol-3-yl)thio]-7(8H)-pteridinone;
2-[[(2,3-difluorophenyl)methyl]thio]-6-[(4-hydroxycyclohexyl)amino]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-(1,3,4-thiadiazol-2-ylthio)-7(8H)-pteridinone;
2-[[(2,3-difluorophenyl)methyl]thio]-6-[[(1S,4R)-4-hydroxy-2-cyclopenten-1-yl]amino]-4-[[(1R)-2-hydroxy-1-methylethyl]amino]-7(8H)-pteridinone;
2-[[(2,3-difluorophenyl)methyl]thio]-4-[[(1R)-2-hydroxy-1-methylethyl]amino]-6-[(3R)-3-hydroxy-1-pyrrolidinyl]-7(8H)-pteridinone;
2-[[(2,3-difluorophenyl)methyl]thio]-6-(3-hydroxy-3-methyl-1-azetidinyl)-4-[[(1R)-2-hydroxy-1-methylethyl]amino]-7(8H)-pteridinone;
6-[(3S)-3-amino-1-pyrrolidinyl]-2-[[(2,3-difluorophenyl)methyl]thio]-4-[[(1R)-2-hydroxy-1-methylethyl]amino]-7(8H)-pteridinone; and
6-[(2-aminoethyl)thio]-2-[[(2,3-difluorophenyl)methyl]thio]-4-[[(1R)-2-hydroxy-1-methylethyl]amino]-7(8H)-pteridinone.

10. A process for the preparation of:
(a) a compound of formula (I) as defined in claim 1 where Y is NR²⁰R²¹ which comprises treatment of a compound of formula (IIA): 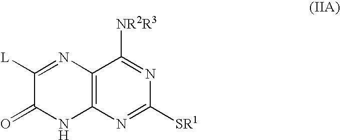
where R¹, R² and R³ are as defined in formula (I) or are protected derivatives thereof and L is a leaving group such as bromo with an amine HNR²⁰R²¹, or
(b) a compound of formula (I) as defined in claim 1 where Y is OR⁴ which comprises treatment of a compound of formula (IIA) where R¹, R² and R³ are as defined in formula (I) or are protected derivatives thereof and L is a leaving group such as bromo with an alcohol R⁴OH, or
(c) a compound of formula (I) as defined in claim 1 where Y is SR⁴ which comprises treatment of a compound of formula (IIA) where R¹, R² and R³ are as defined in formula (I) or are protected derivatives thereof and L is a leaving group such as bromo with a thiol R⁴SH, or
(d) a compound of formula (I) where Y is NR⁵R⁶which comprises treatment of a compound of formula (IIA) where R¹, R² and R³ are as defined in formula (I) or are protected derivatives thereof and L is a leaving group such as bromo with an amine H NR⁵R⁶, or
(e) a compound of formula (I) where Y is a heteroaryl group which comprises treatment of a compound of formula (IIA) where R¹, R² and R³ are as defined in formula (I) or are protected derivatives thereof and L is a leaving group such as bromo with a heteroarene, or
(f) a compound of formula (I) as defined in claim 1 where Y is OH which comprises treatment of a compound of formula (IIB): 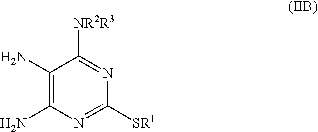
where R¹, R² and R³ are as defined in formula (I) or are protected derivatives thereof with diethyl oxalate, or
(g) a compound of formula (I) as defined in claim 1 where Y is NH₂ which comprises treatment of a compound of formula (IIB) where R¹, R² and R³ are as defined in formula (I) or are protected derivatives thereof with iminomethoxy-acetic acid, methyl ester hydrochloride, and optionally thereafter process (a), (b), (c), (d) or (e) and in any order:
removing any protecting groups
forming a pharmaceutically acceptable salt, solvate or in vivo hydrolysable ester.

11. An intermediate compound of formula (IIA) as defined in claim 10.

12. A pharmaceutical composition comprising a compound of formula (I), or a pharmaceutically acceptable salt, solvate or in vivo hydrolysable ester thereof, as claimed claim 1 in association with a pharmaceutically acceptable adjuvant, diluent or carrier.

13. A process for the preparation of a pharmaceutical composition as claimed in claim 12 which comprises mixing a compound of formula (I), or a pharmaceutically acceptable salt, solvate or in vivo hydrolysable ester thereof, as claimed in claim 1 with a pharmaceutically acceptable adjuvant, diluent or carrier.

14. A compound of formula (I), or a pharmaceutically-acceptable salt, solvate or in vivo hydrolysable ester thereof, as claimed in claim 1 for use in therapy.

15.-16. (Cancelled).

17. A method of treating a chemokine mediated disease wherein the chemokine binds to one or more chemokine receptors, which comprises administering to a patient a therapeutically effective amount of a compound of formula (I), or a pharmaceutically acceptable salt, solvate or in vivo hydrolysable ester thereof, as claimed in claim 1.

18. A method according to claim 17 in which the chemokine receptor belongs to the CXC chemokine receptor subfamily.

19. A method according to claim 17 in which the chemokine receptor is the CXCR2 receptor.

20. A method of treating an inflammatory disease in a patient suffering from, or at risk of, said disease, which comprises administering to the patient a therapeutically effective amount of a compound of formula (I), or a pharmaceutically acceptable salt, solvate or in vivo hydrolysable ester thereof, as claimed in claim 1.

21. A method according to claim 20, wherein the disease is psoriasis, rheumatoid arthritis, a disease in which angiogenesis is associated with raised CXCR2 chemokine levels, or COPD.

22. A method according to claim 20, wherein the disease is rheumatoid arthritis.

23. A method according to claim 20, wherein the disease is COPD.
